(19) United States
(12) Patent Application Publication
(10) Pub. No.: US 2015/0297340 A1
(43) Pub. Date: Oct. 22, 2015

(54) CORNEAL IMPLANT DELIVERY DEVICES AND METHODS OF USE

(71) Applicants: Nicolas ESGUERRA, Irvine, CA (US); Adam ARIELY, San Diego, CA (US); Alan Ngoc LE, Lake Forest, CA (US); John KILCOYNE, Lake Forest, CA (US); Jose GUELL, Lake Forest, CA (US); Keith HOLLIDAY, Lake Forest, CA (US)

(72) Inventors: Nicolas ESGUERRA, Irvine, CA (US); Adam ARIELY, San Diego, CA (US); Alan Ngoc LE, Lake Forest, CA (US); John KILCOYNE, Lake Forest, CA (US); Jose GUELL, Lake Forest, CA (US); Keith HOLLIDAY, Lake Forest, CA (US)

(21) Appl. No.: 14/688,226

(22) Filed: Apr. 16, 2015

Related U.S. Application Data
(60) Provisional application No. 61/980,504, filed on Apr. 16, 2014.

(51) Int. Cl. A61F 2/14

(52) U.S. Cl. CPC A61F 2/148

(57) ABSTRACT

Corneal implant inserters and methods of use. The inserters are adapted such that they can be used to deliver corneal inlays into corneal pockets.

### CROSS REFERENCE TO RELATED APPLICATIONS

This application claims the benefit of U.S. Provisional Application No. 61/980,504, filed Apr. 16, 2014, which is incorporated by reference herein.

This application is related to the following applications, the disclosures of which are incorporated herein by reference: U.S. Pat. No. 8,162,953, issued Apr. 24, 2012; U.S. Pub. No. 2013/0253527, published Sep. 26, 2013; and U.S. Pub. No. 2013/0123916, published May 16, 2013.

### INCORPORATION BY REFERENCE

All publications and patent applications mentioned in this specification are herein incorporated by reference to the same extent as if each individual publication or patent application was specifically and individually indicated to be incorporated by reference.

### BACKGROUND

Delivery devices have been described for delivering ophthalmic devices such as intraocular lenses and corneal implants into the eye. Alternative designs and methods of use are needed that can easily and accurately deliver corneal implants, such as a, without limitation, small diameter, hydrophilic, corneal implants.

### SUMMARY

One aspect of the disclosure is a corneal inlay inserter comprising a distal region, the distal region having at least one aperture through at least one of an anterior and a posterior surface thereof; and a fluid channel in fluid communication with the at least one aperture.

In some embodiments the inserter comprises first and second devices adapted and configured to stably interface one another, wherein the fluid channel is within the first device, the second device comprising the distal region having the at least one aperture through an anterior or a posterior surface thereof. The distal end can have a plurality of apertures through the anterior or posterior surface.

In some embodiments the inserter comprises an elongate body comprising the distal region and the fluid channel therein. The distal region can have a plurality of apertures through the anterior or posterior surface. The fluid channel can extend from a proximal end of the elongate body to the at least one aperture.

In some embodiments the inserter further comprises a corneal inlay secured over the at least one aperture.

In some embodiments the distal region does not have any apertures through the posterior surface thereof.

In some embodiments the distal region does not have any apertures through the anterior surface thereof.

In some embodiments the distal region has at least one aperture through the anterior surface and at least one aperture through the posterior surface, the two apertures being in fluid communication.

One aspect of the disclosure is a method of delivering a corneal implant into the eye, comprising: providing a corneal inserter comprising a distal region, the distal region having at least one aperture through at least one of an anterior and a posterior surface thereof, a corneal implant secured to the inserter over the at least one aperture, and a fluid delivery channel in fluid communication with the at least one aperture; and delivering a fluid through the fluid delivery channel and through the at least one aperture to repel the corneal implant from the distal region and onto corneal tissue.

In some embodiments the inserter comprises a first device comprising the distal region, the method further comprising, prior in time to delivering the fluid, securing a second device with a fluid reservoir to the first device. Securing the second device to the first device can comprise securing the second device to a proximal end of the first device. Securing the second device to the first device can comprise securing the second device to a posterior side of the first device. The method can further comprise advancing the first device into a corneal pocket so that the posterior side is closer to the retina than an anterior side of the first device.

In some embodiments the method further comprises advancing the inserter into a corneal pocket before delivering the fluid, wherein delivering the fluid repels the corneal implant into the corneal pocket.

In some embodiments delivering the fluid comprises delivering as little as about 10 microliters of fluid through the fluid delivery channel, as little as 4 microliters, or even as little as 1 microliter, although more fluid may be delivered. In some embodiments the inserter, including the corneal implant, are adapted such that as little as 10 microliters of fluid, or as little as 4 microliters, or even as little as 1 microliter, is all that is needed to be delivered to release the corneal implant from the inserter, even though more fluid can be used. This is in contrast to, for example, intraocular lens delivery systems, which are not adapted so that the intraocular lens can be released or delivered from the delivery device with a relatively very little amount of fluid.

One aspect of the disclosure is a corneal inlay inserter with an elongate body having a fluid delivery channel therein, the fluid delivery channel in fluid communication with a proximal region and at least one aperture extending through at least one of an anterior surface and a posterior surface of a distal region of the elongate body.

In some embodiments the fluid delivery channel is in fluid with a plurality of apertures extending through at least one of the anterior surface and the posterior surface of a distal region of the inserter. The fluid delivery channel can be in fluid communication with at least one aperture extending only through the anterior surface and not the proximal surface. The fluid delivery channel can be in fluid communication with at least one aperture extending only through the posterior surface and not the anterior surface.

In some embodiments the inserter further comprises a corneal inlay secured to the inserter in a position over the at least one aperture on one of the anterior surface and the posterior surface. The corneal inlay can be a hydrophilic inlay, which in any of the embodiments herein can allow for the fluid delivery of the inlay away from or off of the inserter. The corneal inlay can have a diameter of between 1 mm and 5 mm. The corneal inlay can have a thickness between about 10 microns and about 100 microns.

In some embodiments the corneal implant has a diameter greater than the greatest linear dimension of the at least one aperture measured across the aperture in the proximal to distal direction.

In some embodiments a proximal end of the inserter is configured to interface with a fluid delivery device, the fluid delivery device adapted to advance fluid through the fluid delivery channel and out of the at least one aperture.

In some embodiments the inserter further comprises a securing member positioned over the at least one aperture, the securing member and the distal region defining a volume in which a corneal inlay can be disposed, the securing member configured to be movable relative to the distal region to provide access to the volume.

In some embodiments the at least one aperture has a greatest linear dimension measured across the aperture in the proximal to distal direction of 0.02 mm and 1.0 mm.

In some embodiments the anterior and posterior surfaces are substantially parallel with each other.

One aspect of the disclosure is a method of delivering a corneal implant into the eye, comprising: providing a corneal implant inserter with a fluid delivery channel therein in fluid communication with at least one aperture in at least one of an anterior surface and a posterior surface of a distal region of the inserter, and a corneal implant secured to the inserter over the at least aperture on one of the anterior surface and the posterior surface; and delivering a fluid through the fluid delivery channel and through the at least one aperture to repel the corneal implant from the distal region and into the eye.

In some embodiments the method further comprises securing a fluid reservoir to a proximal end of the inserter, wherein the delivering step comprises delivering fluid from the fluid reservoir and into the fluid delivery channel.

In some embodiments the method further comprises advancing the corneal implant inserter into a corneal pocket before delivering the fluid, wherein delivering the fluid repels the corneal implant into the corneal pocket.

In some embodiments delivering the fluid comprises delivering as little as 10 microliters of fluid, as little as 4 microliters, or even as little as 1 microliter, through the fluid delivery channel, although more may be delivered. In some embodiments the inserter, including the corneal implant, are adapted such that as little as 10 microliters of fluid, as little as 4 microliters, or even as little as 1 microliter, is all that is needed to be delivered to release the corneal implant from the inserter, even though more fluid can be used. This is in contrast to, for example, intraocular lens delivery systems, which are not adapted so that the intraocular lens can be released or delivered from the delivery device with a very little amount of fluid.

One aspect of the disclosure is a corneal inlay inserter comprising a distal region having at least one aperture extending from an anterior surface to a posterior surface, and a hydrophobic member adapted to be moved from a first position to a second position closer to the at least one aperture.

In some embodiments the hydrophobic member is secured indirectly to the distal region but movable relative to the distal region.

In some embodiments the inserter further comprises an actuatable member, the hydrophobic member adapted to be moved closer to the at least one aperture upon actuation of the actuatable member.

One aspect of the disclosure is an ophthalmic device inserter comprising a distal region and an ophthalmic device retained at the distal region, and a repelling member adapted to be moved relative to the ophthalmic device, the repelling member having one or more physical properties adapted to repel the ophthalmic device away from distal region when moved towards the ophthalmic device without making direct physical contact with the ophthalmic device. The ophthalmic device can be hydrophilic.

In some embodiments the ophthalmic device is a corneal implant, the distal region having an anterior surface, a posterior surface, and at least one aperture extending from the anterior surface to the posterior surface, the corneal implant secured to the anterior surface or the posterior surface over at least one aperture; and the repelling member can be adapted to be moved relative to the corneal implant to repel the corneal implant from the anterior surface or posterior surface.

In some embodiments the repelling member is secured indirectly to the distal region but movable relative to the distal region.

In some embodiments the inserter further comprises an actuatable member, the repelling member adapted to be moved closer to the ophthalmic device upon actuation of the actuatable member.

One aspect of the disclosure is a method of delivering an ophthalmic device into the eye, comprising: providing an ophthalmic device and an ophthalmic device inserter, the inserter comprising a distal region and a repelling member having one or more physical properties adapted to repel the ophthalmic device; and repelling the ophthalmic device from the distal region of the inserter and into the eye by moving the repelling member towards the ophthalmic device without making direct physical contact with the ophthalmic device.

In some embodiments the distal region comprises at least one aperture extending from an anterior surface to a posterior surface, the ophthalmic device retained to the anterior surface or the posterior surface over at least one aperture; and wherein repelling the ophthalmic device comprises moving the repelling member closer to the ophthalmic device on the other of the anterior and posterior surfaces.

### BRIEF DESCRIPTION OF FIGURES

FIG. 1 illustrates an exemplary inserter with a fluid channel in fluid communication with a plurality of apertures in an anterior surface of a distal region.

FIG. 2 illustrates the inserter from FIG. 1 attached to a fluid reservoir.

FIG. 3 illustrates an inserter with a fluid channel in fluid communication with one aperture in an anterior surface of a distal region.

FIGS. 4A-4H illustrate an exemplary inserter comprising a first device with a fluid delivery channel therein and a second device with at least one aperture through an anterior surface and a posterior surface. The first and second device are configured to stably interface with each other.

FIGS. 5A-5C illustrate an exemplary inserter that includes an actuatable repelling member adapted to repel the implant away from the inserter and into the eye.

### DETAILED DESCRIPTION

The disclosure herein describes devices that are adapted for positioning ophthalmic devices, such as corneal implants, onto or into corneal tissue. These types of devices may be generally referred to herein as inserters.

Corneal implants can correct vision impairment by creating a change in curvature of the anterior surface of a cornea and/or creating multifocalities within the cornea due to intrinsic properties of the implant. “Corneal implants” as used herein includes corneal onlays and corneal inlays. An onlay is an implant that is placed over the stromal part of the cornea such that the outer layer of the cornea, i.e., the epithelium, can grow over and encompass the implant. An inlay is an implant that is implanted within corneal tissue beneath a portion of the corneal tissue by, for example, cutting a flap in the cornea and inserting the inlay beneath the flap, or by placing it within a pocket created within the cornea. Both inlays and onlays can alter the refractive power of the cornea by changing the shape of the anterior cornea, by having a different index of refraction than the cornea, or both. When the disclosure herein refers to an “inlay,” it is understood that the devices and methods can be used for other types of corneal implants as well.

There is a need for improved devices, systems and methods for inserting corneal implants onto corneal tissue, including inserting them within a corneal pocket.

A corneal “pocket” is generally referred to as a recess formed within the corneal tissue for receiving a corneal implant. Methods of creating and accessing pockets are known, such as may be found described in U.S. Pub. No. 2003/0014042, published Jan. 16, 2003, entitled “Method of Creating Stromal Pockets for Corneal Implants,” U.S. Pub. No. 2013/0253527, published Sep. 26, 2013, which are fully incorporated by reference herein. Pockets can be made by, for example, a Femtosecond laser or a Blade Pocket Maker. Additional exemplary methods and devices for creating corneal pockets, or corneal channels, can be found in U.S. Pub. No. 2012/0046680, filed Aug. 23, 2010, the disclosure of which is fully incorporated by reference herein. Any techniques for creating and accessing pockets can be used to create pockets described herein.

Exemplary devices and methods for positioning inlays into corneal pockets can be found described in U.S. Pat. No. 8,162,953 (see, e.g., FIGS. 7-11 and the descriptions thereof). In U.S. Pat. No. 8,162,953 the delivery device includes a holding space at a distal end thereof adapted to house an inlay, and fluid is used to deploy the inlay from the holding space and into the pocket. Additional exemplary devices and methods for positioning inlays in pockets by delivering fluid through a delivery device with a holding space can be found described in U.S. Pub. No. 2013/0253527.

In some additional embodiments devices and methods can use selective adhesion forces between the inlay and the device. For inlays made primarily of a hydrogel material and of small size (such as some of the inlays described in U.S. Pub. No. 2011/0218623, published Sep. 8, 2011), relatively strong forces act on the fluid within the inlay. These embodiments make use of these characteristics of the inlay and the adhesion forces seen between a fluid and various surface geometries. Selective adhesion or transfer of the inlay during different stages of the delivery process can be manipulated by using different material and/or surface geometries. Examples of delivery devices and methods that utilize selective, or “preferential,” adhesion can be found in U.S. Pub. No. 2013/0123916 published May 16, 2013 (see, e.g., relative adhesion between “moderate” and “minimal” bodies, which may be referred to herein as “fine mesh” and “course mesh” materials). Embodiments in U.S. Pub. No. 2013/0123916, for example, are described primarily as being used to deposit an inlay on a corneal bed after a flap has been created and lifted. In U.S. Pub. No. 2013/0123916 a preferential adhesion between two materials is controlled in different stages of delivery until the inlay contacts the stromal bed and adheres to it. In a procedure that delivers an inlay into a pocket, tissue and/or liquid in the eye are constantly contacting the inlay and device material as the device and inlay are advanced towards and into the pocket. Thus, preferential adhesion of the inlay is preferably controlled such that there is a strong attraction between the inlay and the delivery device during insertion into the pocket and a reduction or transfer mechanism such that once the inlay is ready for transfer by position or such, the user can selectively transfer the inlay into the corneal pocket.

One aspect of the disclosure is a corneal inlay inserter comprising a distal region, the distal region having at least one aperture through at least one of an anterior and a posterior surface thereof; and a fluid channel in fluid communication with the at least one aperture.

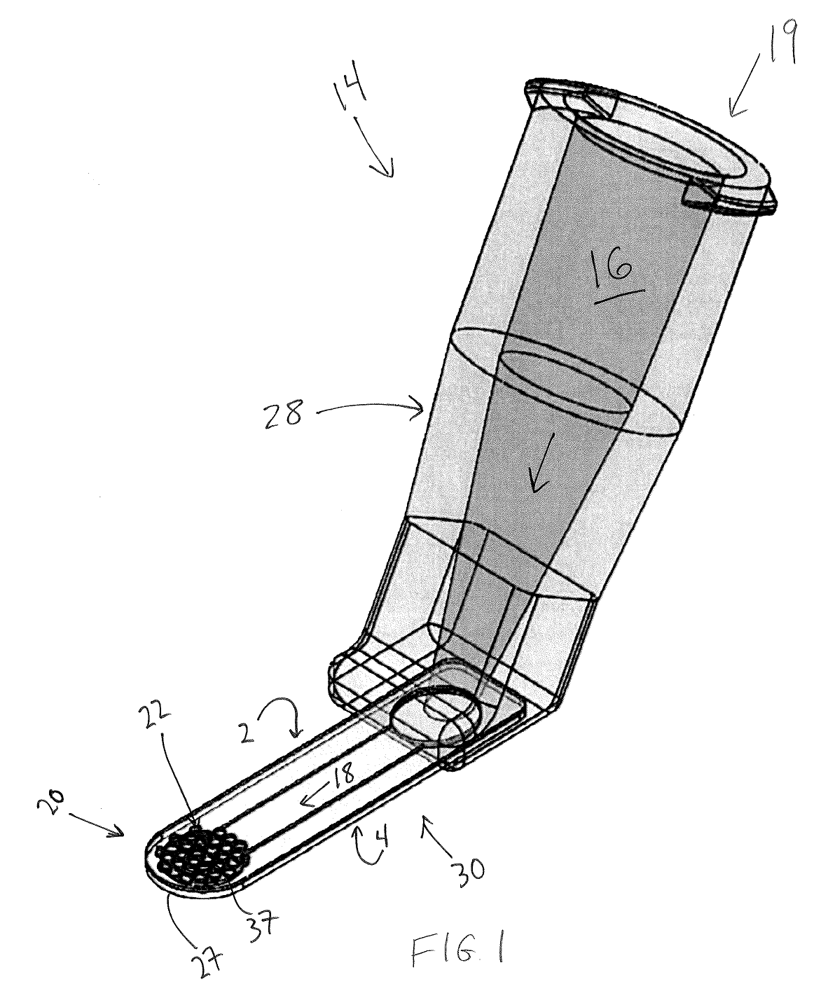

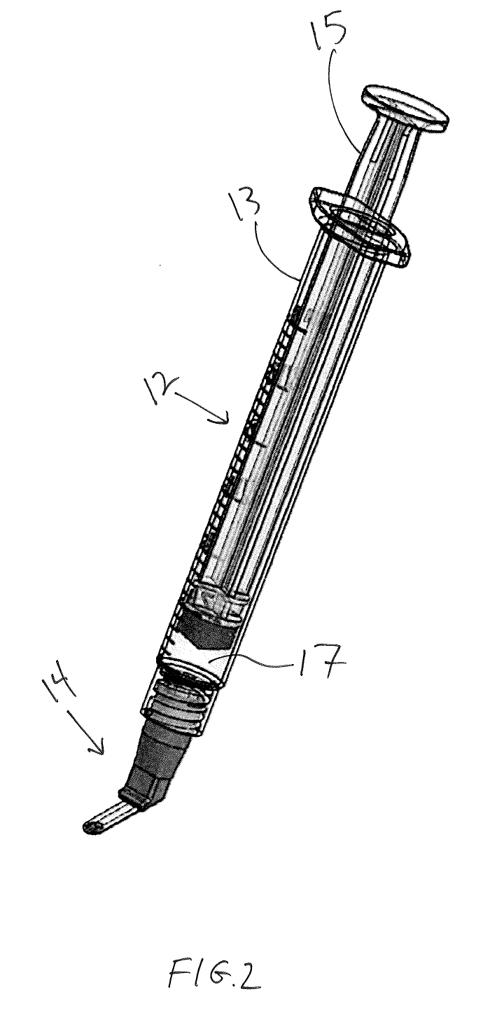

FIGS. 1 and 2 illustrate an example of an inserter 14, which includes a distal region, the distal region having at least one aperture through at least one of an anterior and a posterior surface thereof; and a fluid channel in fluid communication with the at least one aperture. In this embodiment inserter 14 has a proximal region 19 with a proximal end, the proximal region 19 configured to be secured to a fluid reservoir, but is other embodiments the inserter can be integral with the fluid reservoir (i.e., not configured to be detachable without physical deformation or breaking). In this embodiment fluid reservoir 12 (see FIG. 2) is a syringe, which includes fluid chamber 17. The connection between inserter 14 and fluid reservoir 12 can be any suitable method (such as a luer fitting).

Inserter 14 has an elongate body 30 with a distal region 20, the distal region 20 including at least one aperture through at least one of an anterior and a posterior surface of the distal region. Distal region 20 includes anterior surface 2 and posterior surface 4. Anterior and posterior refer to relative positions of the inserter when it is in use. In this context anterior refers to a position closer to the anterior surface of the cornea than the retina, and posterior refers to a relative position closer to the retina than the anterior surface of the cornea. In this embodiment anterior surface 2 may be thought of as a “top” surface, and posterior surface 4 may be thought of as a “bottom” surface of distal region 20.

Inserter 14 also includes fluid channel 18. In this embodiment fluid inserter 14 includes elongate body 30, which includes fluid channel 18 therein in communication with the at least one aperture 22. Fluid channel 18 extends through elongate body 30 to a location directly below, or posterior to, the at least one aperture. In this embodiment distal region 20 has a plurality of apertures 22 in anterior surface 2, and no apertures in posterior surface 4. In some embodiments the distal region has at least one aperture in the posterior surface and none in the anterior surface, while in some embodiments distal region 20 has at least one aperture in each of the anterior surface and the posterior surface.

In this embodiment inserter 14 includes fluid reservoir adaptor 28, which is not integral with elongate body 30 but rather is secured thereto. In alternative embodiments adaptor 28 is integral with elongate body 30, and is considered an extension of elongate body 30 in the general anterior direction. In this embodiment adaptor 28 includes the proximal region 19 that is adapted to be secured to fluid reservoir 12. Adaptor 28 includes fluid channel 16 therein in fluid communication with fluid channel 18 in elongate body 30. When fluid reservoir 12 secured to inserter 14, fluid chamber 17, fluid channel 16, fluid channel 18, and the at least one aperture 22 are all in fluid communication. In embodiments in which adaptor 28 is integral with elongate body 30, fluid channel 16 is simply an extension of fluid channel 18 towards proximal region 19.

In this embodiment adaptor 28 is secured to elongate body 30. Elongate body 30 can be secured to the posterior, or bottom, of adaptor 28 using any number of techniques such as an adhesive. Adaptor 28 can include a receiving portion that is configured to receive the proximal end of elongate body 30 therein.

In this embodiment the inserter is configured to retain an ophthalmic device, in this embodiment a corneal inlay, on anterior side 2 of distal region 20 over the at least one aperture in anterior side 2. In this embodiment distal region 20 includes a plurality of aperture in the anterior surface 2 of distal region 20. In this embodiment the inlay is retained on anterior side 2 on at least some of the plurality of apertures due to adhesion forces. In this embodiment the plurality of apertures 22 functions similarly to a “moderate body” mesh in U.S. Pub. No. 2013/0123916, also referred to as “preferential material”). The distal end 20 is thus configured so that the corneal inlay adheres to it, and is retained by it. Whereas in U.S. Pub. No. 2013/0123916 the corneal inlays are generally described as adhering to a “posterior” side of the moderate body for placement onto the corneal bed, in this embodiment the corneal inlay is disposed positioned on anterior side 2 of the plurality of apertures and is retained thereon.

In the embodiments herein, unless indicated otherwise, the anterior and posterior surfaces are substantially parallel with each other. Substantially parallel does not require them to be precisely parallel, but upon inspection one of ordinary skill in the art would understand them to be substantially parallel. For example, the two surfaces extending proximally to distally in the figures herein are all substantially parallel. The surfaces are substantially parallel even if there is a slight degree of curve to them.

While a “minimal” or “course” mesh material (as described in U.S. Pub. No. 2013/0123916), or any other material that has less preferential adhesion for the corneal inlay than distal end 20, is not shown in the embodiment in FIGS. 1 and 2, it can be assumed that the corneal inlay could in some embodiments be positioned between a fine mesh and a course mesh for storage or packaging, and the course mesh could then be moved relative to the fine mesh to provide access to the inlay, as is described in U.S. Pub. No. 2013/0123916. For example, a packaging and storage device could include both fine and course mesh materials, and the course mesh material is moved away from the fine mesh, and the inlay will preferentially adhere to the fine mesh. Thus, the preferential adhesion principals described in U.S. Pub. No. 2013/0123916 can be utilized in this embodiment, or any of the embodiments herein.

Distal region 20, including the region that defines the apertures, can be, for example without limitation, titanium. Additionally, any of the mesh configuration described in U.S. Pub. No. 2013/0123916 can be used for the mesh configuration of distal region 20.

In alternative embodiments the inlay can be retained on proximal side 4 of inserter 14. For example, in some applications it may be desired to position the corneal implant on the posterior side 4 for placement in the eye. In those embodiments inserter can include a plurality of apertures in proximal side 4 that are in fluid communication with fluid channel 18. There may be added benefits to having at least one aperture on the side of the distal region 20 opposite the side on which the corneal implant is retained. The opposite side can thus have at least one aperture therein in fluid communication with fluid channel 18 even though the implant is not on that side.

In this embodiment the fluid reservoir can be secured to inserter 14, and then held by a user when it is time to advance the implant 37 onto corneal tissue, such as into a pocket. In an exemplary method of use for delivery into a corneal pocket, once inserter 14 is prepared for insertion, the user will introduce distal end 20, on which the corneal inlay is adhered, into the already prepared corneal pocket. Once the desired inlay location is achieved, the user will actuate plunger 15 (which can be any other actuation mechanism) to advance fluid from the reservoir chamber 17, through fluid channels 16 and 18, and out of apertures 22 in the anterior direction. The flow of fluid out of the apertures 22 causes, either hydraulically and/or through the reduction of adhesion forces between the corneal inlay and the distal region 20, repels the inlay away from the distal region 20, separating it from the distal region and thus delivering the inlay into the corneal pocket. The inserter is then removed from the pocket. Any of the inserters herein can also be used, or modified to be used, to deliver the ophthalmic device on a corneal bed formed by creating a corneal flap, or in any other suitable delivery procedure.

In some embodiments inserter 14 is packaged and stored with a corneal implant (such as with two mesh materials in place to secure the implant, as described in applications incorporated herein by reference), and then attached to the fluid reservoir when the inlay is ready for use.

Distal end 20 of inserter 14 can be formed by securing a top, or anterior, piece of material the plurality of apertures formed therein, to a bottom piece with a channel formed therein. When the two pieces are secured together, fluid is directed down now formed fluid channel 18 towards the apertures in the direction of the arrows as shown in FIG. 1. The distal region 20 of inserter 14 can be manufactured in other ways to create fluid channel 18 as well. The exemplary rounded distal end 27 of distal portion 20 is closed so that fluid can only escape the distal end through the apertures, which helps the inlay disassociate from the distal region 20. In some embodiments, however, there may be advantage to having apertures on both sides. In this embodiment the fluid thus acts to break the adhesion between the implant and the distal region 20 and the inlay drifts off of and away from distal region.

Any of the fine mesh materials (also referred to as moderate materials), the orientation of the apertures, and techniques for manufacturing them described in U.S. Pub. No. 2013/0123916 can be used in making the distal region 20 and/or the elongate body 30, or any distal region herein.

In alternative embodiments to that shown in FIGS. 1 and 2, the device can also be adapted so that the implant adheres to the posterior side, or “bottom” of the distal region. The fluid channel could be on top of, or anterior to, the implant, and the fluid would displace the implant from the mesh in the downward, or posterior, direction. Any of the devices herein can be adapted so that the inlay is positioned on either the anterior side or the posterior side of the distal region.

The apertures herein on the anterior side and/or posterior side of the distal region are differentiated from distal ports, through which intraocular lenses or other ophthalmic devices are commonly pushed through during delivery into the eye.

In some embodiments inlay 37 (or any inlays used with any of the inserters herein) has a diameter of between 1 mm and 5 mm. In some embodiments the inlay has a central thickness between about 10 microns and about 100 microns. In some embodiments herein the inlay has a water content of least 60%, and is comprised of a hydrogel. As can be seen in FIG. 1 and in the applications incorporated by reference herein, the diameter of the inlay is greater than the greatest linear dimension of the at least one aperture measured across the aperture in the proximal to distal direction. In any of the embodiments herein the at least one aperture, such as all of them, has a greatest linear dimension measured across the aperture in the proximal to distal direction of between 0.02 mm and 1.0 mm, such as between 0.02 mm and 0.75 mm.

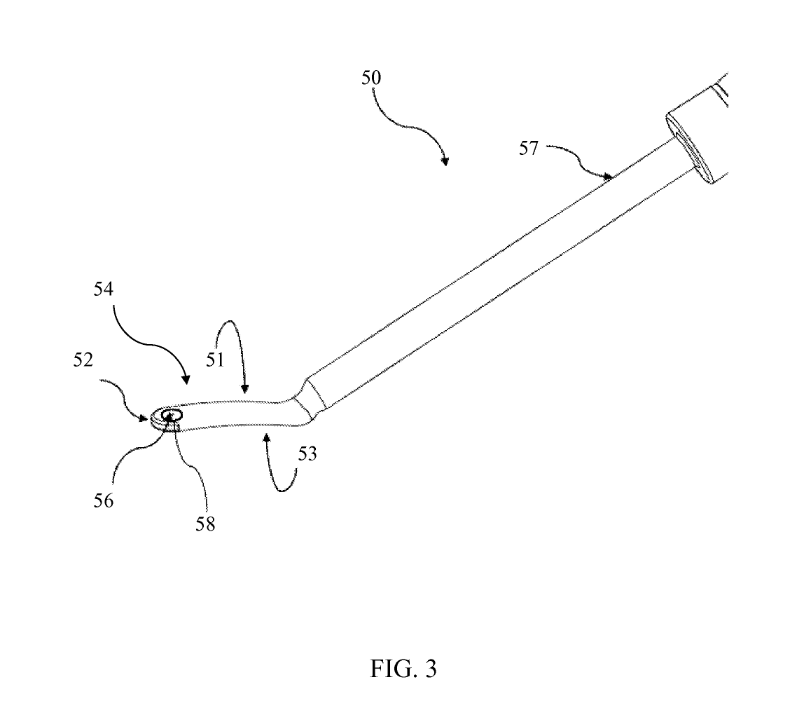

FIG. 3 illustrates an alternative inserter 50 that is adapted to position implant 58 within a corneal pocket. Inserter 50 is similar to common cannulas, but has a generally flattened elongate body 54. Inserter 50 includes elongate body 54 and distal region 52, wherein distal region 52 has only one aperture 56 therein on anterior side 51 and does not have any apertures on posterior side 53. This is an example of at least one aperture but not more than one. A fluid channel (not labeled in FIG. 3) extends through inserter 50 from aperture 56 through elongate body 54 and into handle portion 57. Fluid can be advanced through the fluid channel (not shown) and out of aperture 56 using known techniques, such as with a plunger. Anterior surface 51 and posterior surface 53 are substantially parallel in this embodiment as well.

In an exemplary method of use, inlay 58, once positioned on distal region 52 over aperture 56, adheres to distal region 52 due to some adhesion forces. The adhesion forces may not be as great as those present in the embodiment in FIGS. 1 and 2, however. The inserter, with the inlay adhered thereto, is then advanced into a corneal pocket as described herein. To dissociate inlay 56 from inserter 50, fluid is advanced through the fluid channel and out of aperture 56, causing the inlay to be displaced from the inserter 50 and into the pocket. Again, inserter 50 could have the aperture(s) on the proximal side 53 such that the inlay is displaced from inserter 50 in a downward, or posterior, direction.

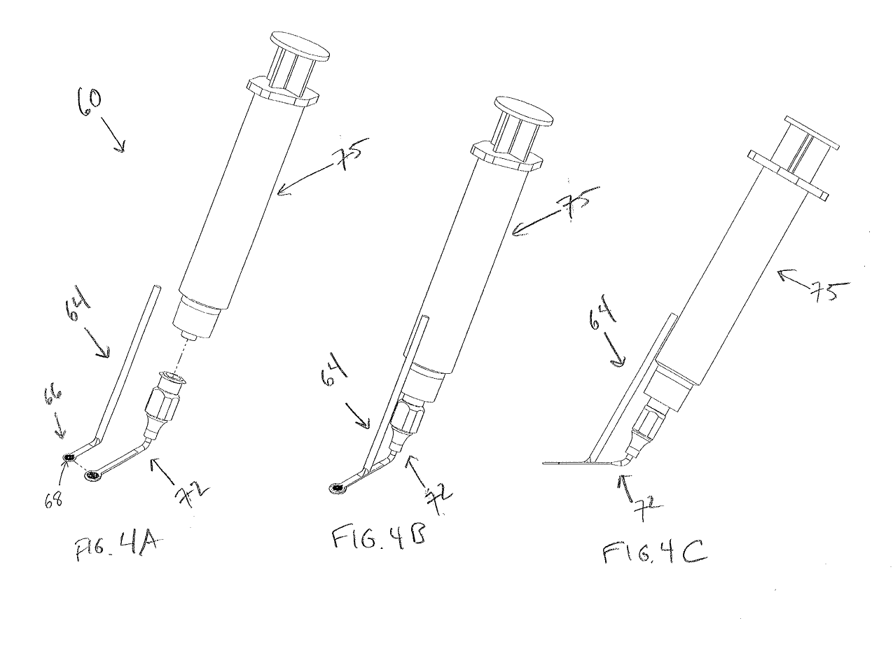

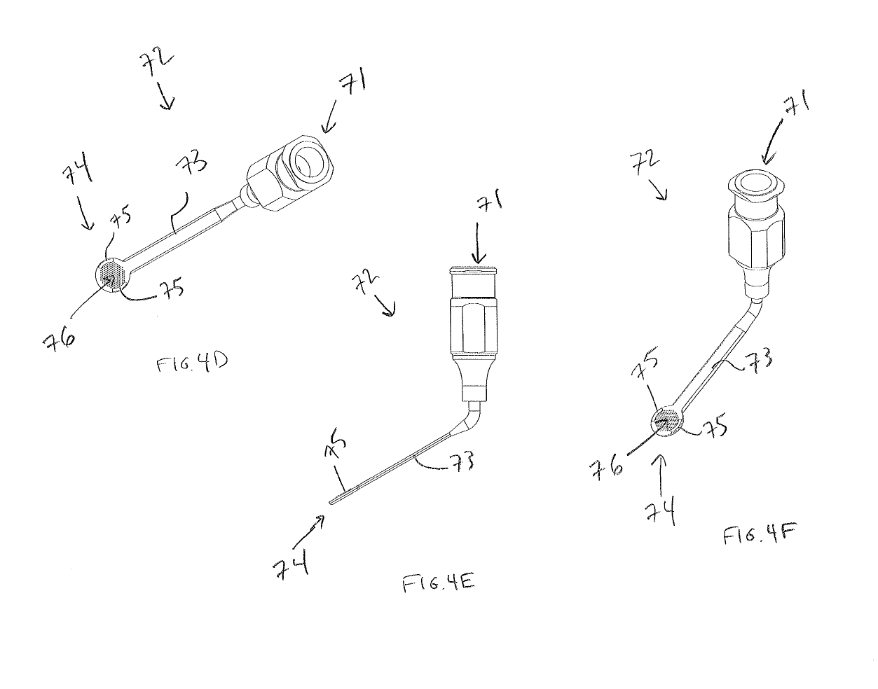

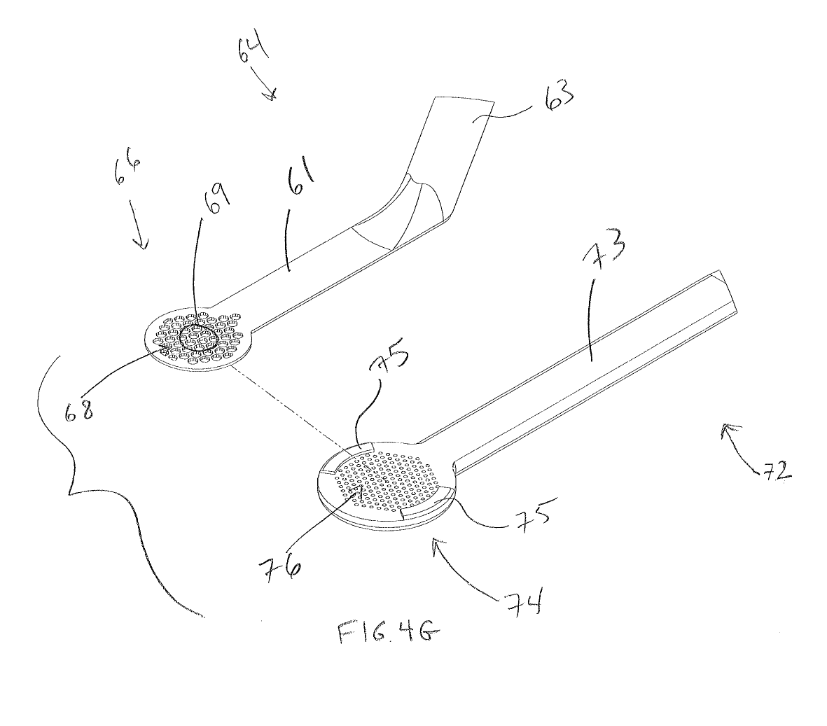

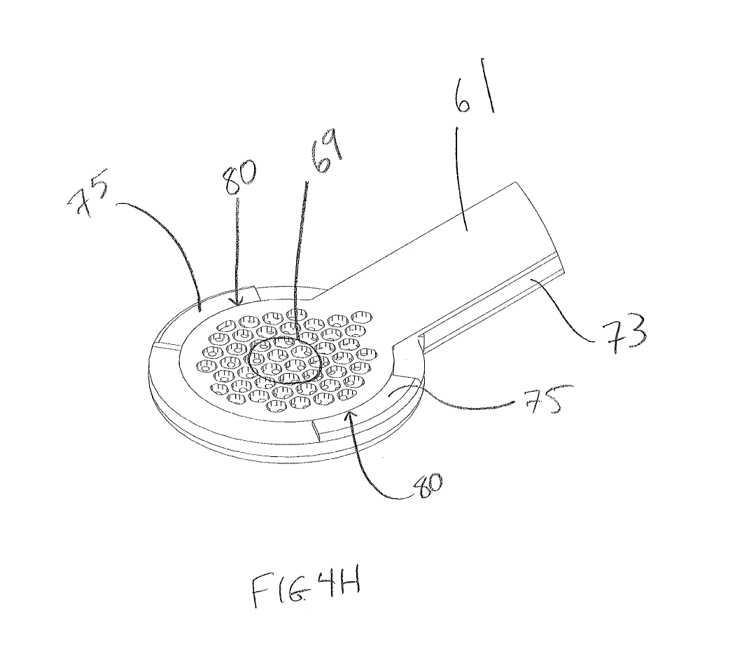

The embodiment shown in FIGS. 4A-4H is an example of an inserter that includes a distal region, the distal region having at least one aperture through an anterior surface and a posterior surface thereof, and a fluid channel in fluid communication with the at least one aperture. In this embodiment inserter 60 comprises first 72 and second 64 separate devices that are adapted and configured to stably interface one another, wherein the fluid channel is within first device 72, and second device 64 comprises distal region 66 having the at least one aperture 68 through the anterior and posterior surfaces thereof. The fluid channel (not shown for clarity) is thus in fluid communication with the at least one aperture 68 when the first 72 and second 64 devices and secured to one another. In this embodiment the second device 64 can be a mesh device such as any of the meshes described herein, and can be used in any of the methods described herein. For example, a corneal implant can be secured to an anterior surface of the distal region of the mesh (e.g., a fine mesh) due to adhesion forces.

FIGS. 4A-4H illustrate an exemplary inserter 60 that includes first device 72 with distal region 74 that includes side ridges 75 at the periphery of distal region 74 positioned and configured so that distal region 74 of first device 72 can stably interface distal region 66 of second device 64 in at least one direction. Second device 64 includes a fine mesh configuration described herein, and a corneal implant 69 secured thereon (see FIG. 4G). FIGS. 4B, 4C, and 4H illustrate the inserter after the first and second devices are stably interfacing each other. First device 72 of the inserter is configured so that it can be attached at its proximal end 71 to a fluid reservoir 75 (FIGS. 4A-4C), such as a syringe with plunger, with the luer lock.

FIG. 4A shows the exemplary inserter, including first device 72, second device 64, and fluid reservoir 75. FIG. 4B is a perspective view showing inserter 60 after first device 72 and second device 64 are stably interfacing, and after reservoir 75 has been secured to first device 72. FIG. 4D shows a top view of first device 72. FIG. 4E shows a side view of first device 72, and FIG. 4F shows a perspective view of first device 72. FIG. 4G shows the distal regions 66 and 74, of second device 64 and first device 72, respectively, not in an interfacing relationship. FIG. 4H is a close-up perspective view of the distal regions 66 and 74 in an interfacing relationship. The first device interfaces the second device at interface regions 80, thereby stabilizing the second device 64 relative to the first device 72 in at least one direction.

In use, after the first and second devices are secured to one another (such as shown in FIGS. 4B, 4C, and 4H), fluid is delivered through the fluid channel in first device 72 (e.g., using a syringe in fluid reservoir 75), out of the apertures 76 in the second device 72, and then through the mesh aperture(s) 68 in second device 64, causing the corneal implant 69 to be disassociated from (i.e., drift off of) distal region 66 of second device 64, as is described elsewhere herein.

FIGS. 4C and 4H show first device 72 with fluid channel therein positioned posterior to the mesh of first device 64 and inlay. In some embodiments first device 72 includes a fluid pillow at its distal end. In these embodiments, as fluid is delivered from the syringe to the fluid (e.g., saline) pillow, fluid is delivered through the mesh apertures more gently due to the presence of the pillow. Thus the pillow can be used to advance fluid through the mesh aperture in a more controlled and gentle manner. In this embodiment the pillow can be a perforated device, similar in concept to a teabag.

It is understood that other types of mechanisms can be used to secure the first and second device together and still fall within the subject matter of this disclosure. For example, the distal region of the second device 64 can be placed under (posterior to) restraining clips in the distal region of the first device 72.

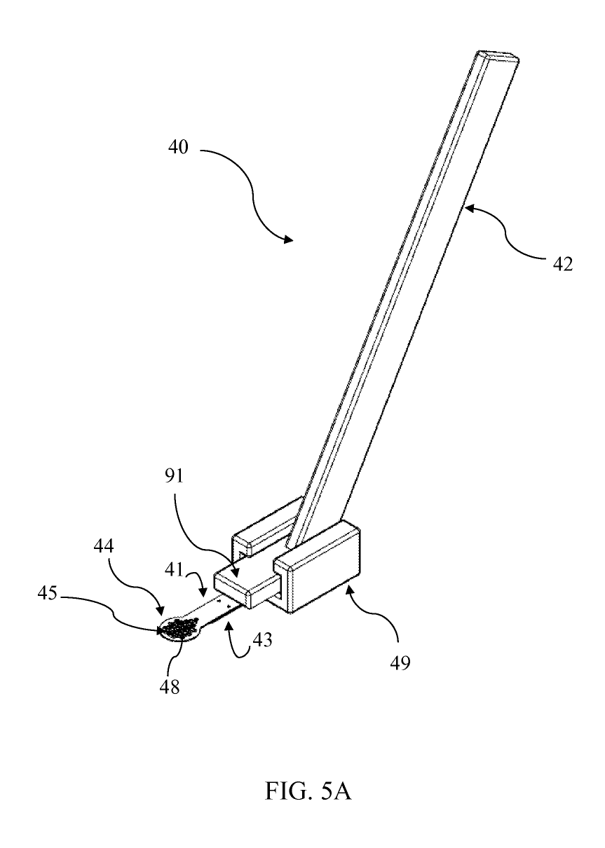

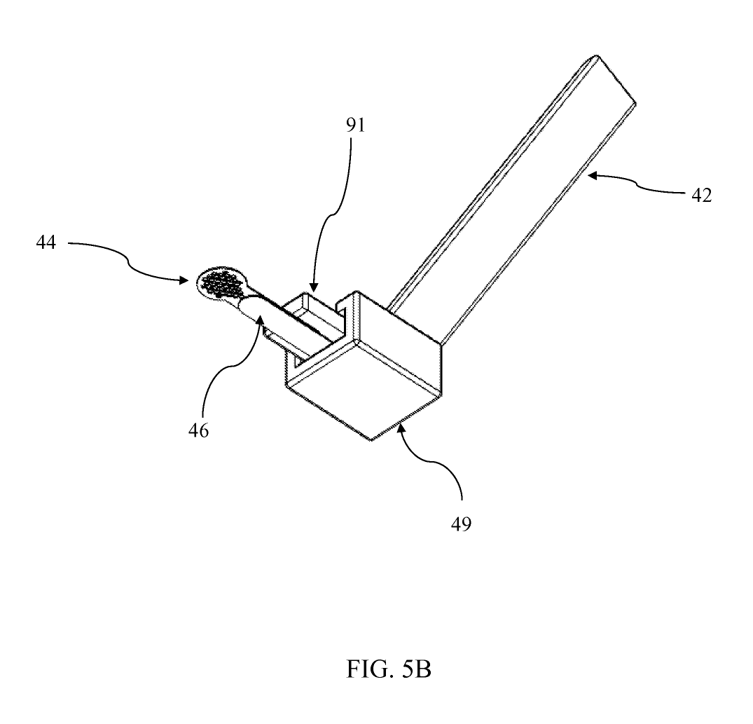

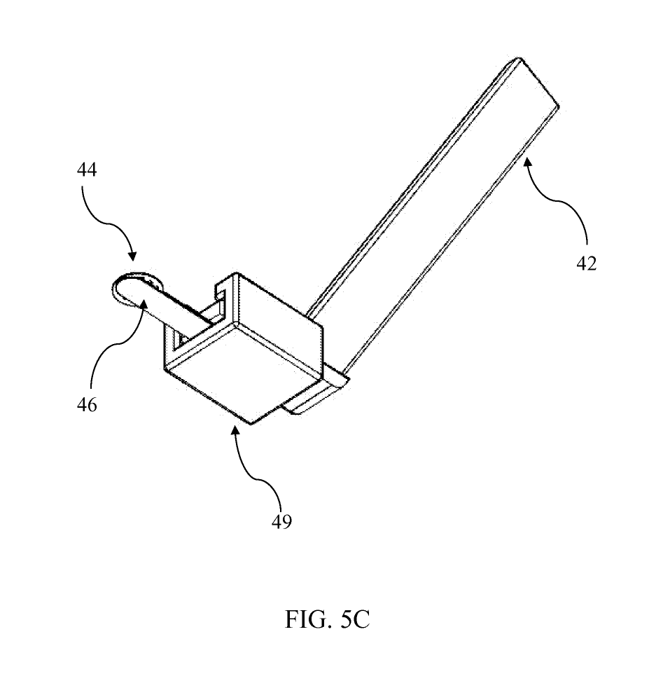

In some embodiments herein an ophthalmic device inserter comprises a distal region and an ophthalmic device retained at the distal region, and a repelling member adapted to be moved relative to the ophthalmic device, the repelling member having one or more physical properties that cause it to repel the ophthalmic device away from the distal region when moved towards the ophthalmic device without making direct physical contact with the ophthalmic device. The inserter and ophthalmic device in FIGS. 5A-5C is an example of such an inserter and ophthalmic device.

Inserter 40 includes a handle portion 42 secured to distal region 44. Inserter 40 also includes an actuatable moveable member 49 that includes repelling member 46 (see FIGS. 5B and 5C) extending distally from the base of actuatable member 49. Distal region 44 is coupled to handle 42 via connector 91, to which distal region 44 is secured.

Distal region 44 can be considered very similar or even the same as the mesh materials (moderate or minimal) incorporated by reference herein. Distal region includes a plurality of apertures 45 therethrough, from anterior surface 41 to proximal surface 43. Ophthalmic device 48 and distal region 44 are adapted such that ophthalmic device 48 is retained on distal region 44 due to adhesion forces. Rather than simply using fluid flow to deliver the implant, however, in this embodiment a repelling member is brought into closer proximity to the implant, which causes the implant to be repelled from distal region 44 and into the eye. In this exemplary embodiment inserter includes an actuable member 49 that is coupled to repelling member 46, and when actuated repelling member 46 is moved closer to implant 48 along the posterior surface 43 of distal region 44. In some embodiments in which the implant has a high enough water content, repelling member 46 is a hydrophobic material that when moved into closer proximity of the implant, it repels the implant away from distal end 44 in the anterior direction, away from distal end 44 and into the eye. FIG. 5C shows a posterior view of repelling member 46 after it has been moved into closer proximity to implant 48, relative to an initial position shown in FIG. 5B prior to actuation. In FIG. 5C, repelling member is disposed on the posterior side of at least some of the apertures 45. In alternative embodiments the implant is secured to posterior side of the distal region, and the inserter is constructed and arranged such that the repelling member moves into closer proximity to the implant on the anterior side. In these embodiments the implant is repelled away from the distal end in the posterior direction into the eye.

In some embodiments the repelling member is a hydrophobic material that, when moved into closer proximity to the implant, will repel the fluid and/or the implant away from the hydrophobic material, thus deploying the implant from the inserter and into the pocket. A material such as Teflon can be used as the hydrophobic material. Other hydrophobic materials can also be used, however. In this embodiment the implant is adhered to one material that can be considered hydrophilic, while repelling member 46 is hydrophobic. Repelling member 46 can alternatively be actuated in any conceivable way towards the implant/distal region interface, such as via an actuator on the handle that causes the actuatable member 49 to move.

The repelling action in embodiments in which the repelling member is a hydrophobic material is at least partially based on the lotus effect, named after the lotus leaf. The lotus effect refers generally to self-cleaning properties that are result of very high water repellence (superhydrophobicity), as exhibited by the leaves of the lotus flower. Part of the reason the lotus leaf is so repellent is due to air trapped in its nodule-covered surface. The effect relies on surface tension, therefore there needs to be a surface between air and water. The hydrophobic members herein can have surfaces that are configured (such as through modification) to trap air in order to increase the efficiency of the repelling action.

The embodiment in FIGS. 5A-5C is an example of using a non-fluid member to deliver the implant, and one that does not come into direct contact with the implant to deliver the implant from the inserter.

The embodiment in FIGS. 5A-5C can be used in any of the methods of use herein to deploy the implant in a corneal pocket.

What is claimed is:

1. A corneal inlay inserter comprising a distal region, the distal region having at least one aperture through at least one of an anterior and a posterior surface thereof; and a fluid channel in fluid communication with the at least one aperture.

2. The corneal inlay inserter of claim 1 comprising first and second devices adapted and configured to stably interface with one another, wherein the fluid channel is within the first device, the second device comprising the distal region having the at least one aperture through an anterior or a posterior surface thereof.

3. The corneal inlay inserter of claim 2 wherein the distal region has a plurality of apertures through the anterior or posterior surface.

4. The corneal inlay inserter of claim 1 wherein the inserter comprises an elongate body comprising the distal region and the fluid channel therein.

5. The corneal inlay inserter of claim 4 wherein the distal region has a plurality of apertures through the anterior or posterior surface.

6. The corneal inlay inserter of claim 4 wherein the fluid channel extends from a proximal end of the elongate body to the at least one aperture.

7. The corneal inlay inserter of claim 1 further comprising a corneal inlay secured over the at least one aperture.

8. The corneal inlay inserter of claim 1 wherein the distal region does not have any aperture through the posterior surface thereof.

9. The corneal inlay inserter of claim 1 wherein the distal region does not have any aperture through the anterior surface thereof.

10. The corneal inlay inserter of claim 1 wherein the distal region has at least one aperture through the anterior surface and at least one aperture through the posterior surface, the two apertures being in fluid communication.

11. A method of delivering a corneal implant into the eye, comprising:
providing a corneal inserter comprising a distal region, the distal region having at least one aperture through at least one of an anterior and a posterior surface thereof, a corneal implant secured to the inserter over the at least aperture, and a fluid delivery channel in fluid communication with the at least one aperture; and
delivering a fluid through the fluid delivery channel and through the at least one aperture to repel the corneal implant from the distal region and onto corneal tissue.

12. The method of claim 11 wherein the inserter comprises a first device comprising the distal region, the method further comprising, prior in time to delivering the fluid, securing a second device with a fluid reservoir to the first device.

13. The method of claim 12 wherein securing the second device to the first device comprises securing the second device to a proximal end of the first device.

14. The method of claim 12 wherein securing the second device to the first device comprises securing the second device to a posterior side of the first device.

15. The method of claim 14 further comprising advancing the first device into a corneal pocket so that the posterior side is closer to the retina than an anterior side of the first device.

16. The method of claim 11 further comprising advancing the inserter into a corneal pocket before delivering the fluid, wherein delivering the fluid repels the corneal implant into the corneal pocket.

17. The method of claim 11 wherein delivering the fluid comprises delivering as little as 10 microliters of fluid through the fluid delivery channel.

18. A corneal inlay inserter with an elongate body having a fluid delivery channel therein, the fluid delivery channel in fluid communication with a proximal region and at least one aperture extending through at least one of an anterior surface and a posterior surface of a distal region of the elongate body.

19. The corneal inlay inserter of claim 18 wherein the fluid delivery channel is in fluid with a plurality of apertures extending through at least one of the anterior surface and the posterior surface of a distal region of the inserter.

20. The corneal inlay inserter of claim 19 wherein the fluid delivery channel is in fluid communication with at least one aperture extending only through the anterior surface and not the proximal surface.

21. The corneal inlay inserter of claim 19 wherein the fluid delivery channel is in fluid communication with at least one aperture extending only through the posterior surface and not the anterior surface.

22. The corneal inlay inserter of claim 18 further comprising a corneal inlay secured to the inserter in a position over the at least one aperture on one of the anterior surface and the posterior surface.

23. The corneal inlay inserter of claim 22 wherein the corneal inlay has a diameter of between 1 mm and 5 mm.

24. The corneal inlay inserter of claim 23 wherein the corneal inlay has a thickness between about 10 microns and about 100 microns.

25. The corneal inlay inserter of claim 18 wherein the corneal implant has a diameter greater than the greatest linear dimension of the at least one aperture measured across the aperture in the proximal to distal direction.

26. The corneal inlay inserter of claim 18 wherein a proximal end of the inserter is configured to interface with a fluid delivery device, the fluid delivery device adapted to advance fluid through the fluid delivery channel and out of the at least one aperture.

27. The corneal inlay inserter of claim 18 further comprising a securing member positioned over the at least one aperture, the securing member and the distal region defining a volume in which a corneal inlay can be disposed, the securing member configured to be movable relative to the distal region to provide access to the volume.

28. The corneal inlay inserter of claim 18 wherein the at least one aperture has a greatest linear dimension measured across the aperture in the proximal to distal direction of 0.02 mm and 1.0 mm.

29. The corneal inlay inserter of claim 18 wherein the anterior and posterior surfaces are substantially parallel with each other.

30. A method of delivering a corneal implant into the eye, comprising:
providing a corneal implant inserter with a fluid delivery channel therein in fluid communication with at least one aperture in at least one of an anterior surface and a posterior surface of a distal region of the inserter, and a corneal implant secured to the inserter over the at least aperture on one of the anterior surface and the posterior surface; and
delivering a fluid through the fluid delivery channel and through the at least one aperture to repel the corneal implant from the distal region and into the eye.

31. The method of claim 30 further comprising securing a fluid reservoir to a proximal end of the inserter, wherein the delivering step comprises delivering fluid from the fluid reservoir and into the fluid delivery channel.

32. The method of claim 30 further comprising advancing the corneal implant inserter into a corneal pocket before delivering the fluid, wherein delivering the fluid repels the corneal implant into the corneal pocket.

33. The method of claim 30 wherein delivering the fluid comprises delivering as little as 10 microliters of fluid through the fluid delivery channel.
